(19) United States
(12) Patent Application Publication
(10) Pub. No.: US 2007/0016090 A1
(43) Pub. Date: Jan. 18, 2007

(54) Implantable medical devices and related methods

(75) Inventors: Brian P. Brockway, Shoreview, MN (US); Perry A. Mills, Arden Hills, MN (US); Arthur J. Foster, Centerville, MN (US); Scott Lambert, East Bethel, MN (US); Kathy Lynn Sherwood, North Oaks, MN (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Transoma Medical, Inc., St. Paul, MN (US)

(21) Appl. No.: 11/509,850

(22) Filed: Aug. 25, 2006

Related U.S. Application Data
(63) Continuation-in-part of application No. 11/119,358, filed on Apr. 28, 2005.
(60) Provisional application No. 60/566,222, filed on Apr. 28, 2004.

(51) Int. Cl. A61B 5/04

(52) U.S. Cl. 600/509; 128/903

(57) ABSTRACT

Implantable medical devices and associated methods are disclosed. In one implementation, the implantable medical device comprises a conductive housing and a remote electrode that is mechanically coupled to the conductive housing by a lead body. An amplifier is electrically connected to the remote electrode and the conductive housing for providing a signal representative of a voltage difference between the remote electrode and the conductive housing. In some methods in accordance with the present invention, the implantable medical device is implanted in an implant site overlaying one half of a rib cage of a human body. The implantable medical device produces a signal representative of the voltage difference between the remote electrode and the conductive housing and the signal is transmitted to a receiver located outside the human body.

### CROSS REFERENCE TO RELATED APPLICATION

The present application is a continuation in part of U.S. patent application Ser. No. 11/119,358, filed Apr. 28, 2005, which is hereby incorporated by reference.

### BACKGROUND OF THE INVENTION

Some types of implantable devices provide for measurement of ECG and other information which may be transmitted to an external recorder and/or analysis device. The information thus recorded can be used by a physician or other medical care provider to aid in diagnosis or treatment or for alerting emergency medical services of a life-threatening event. Current systems commercially available for the same or similar purpose include the Reveal® implantable loop recorder (ILR) available from Medtronic (Minneapolis, Minn.), animal monitoring devices available from Data Sciences International (St. Paul, Minn.), mobile outpatient cardiac telemetry systems and services available from Cardionet (San Diego, Calif.), and various hardwired systems.

The Medtronic Reveal is an ECG monitor intended for diagnosis of syncope or other rhythm disturbances. This device analyzes the ECG in real time. The device detects when a rhythm disturbance occurs and stores a segment of the ECG strip before and after the time of the rhythm disturbance. Issues with this include limited signal processing capability leading to poor detection accuracy. This device is often unable to, for example, detect atrial fibrillation accurately. In addition, it often falsely detects rhythm disturbances resulting in ECG's with no useful diagnostic utility filling the memory of the device. Memory in this device is limited to about 40 minutes, and the patient must visit the clinic in order for the memory of the device to be dumped and reset. Once the memory fills, a syncopal event can no longer be recorded. Since these events can occur very infrequently, this can limit the diagnostic utility of the device. The Reveal includes ECG electrodes that are incorporated into the body of the device. One electrode is in the header and the 2nd electrodes is an uninsulated portion located at the opposite end of the metallic body of the device.

The Data Sciences International (DSI) system for monitoring animals involves an implanted ECG, temperature, and pressure transmitter that telemeters a continuous ECG. Information from this device is transmitted in real time to a receiver. The receiver forwards a signal to a computing device where the signals are analyzed (ECGs for arrhythmias, intervals; pressure for systolic, diastolic, and mean pressure, heart rate, dP/dt, etc.) The transmitter employs flexible leads for sensing that extend from the body of the device.

The Cardionet system involves surface electrodes that are placed on the patient for monitoring ECG. The ECG signal is telemetered to a computing device that analyzes the ECG and identifies rhythm abnormalities. This device can forward a real time ECG to a monitoring station, or can notify the monitoring station if an abnormal rhythm is identified. This system packetizes the telemetered signal, incorporates time synchronization, and the receiver identifies whether a particular packet was received properly. If a packet was not received properly, the computing device signals to the transmitter to resend a packet. This device requires that surface electrodes be worn. Wires from the surface electrodes are connected to the telemetry device worn by the patient. This can particularly be a problem while the patient is sleeping. Also, since surface electrodes must be worn, patient compliance is an issue. Most patients are unwilling to wear surface electrodes for more than about three to four weeks. This system provides the advantage of real time monitoring can be accomplished. If the surface electrodes come loose, this can be identified immediately by the monitoring center and the patient can be contacted to reposition the electrodes.

Hardwired systems are available to serve this purpose. A computing device connects directly to surface electrodes for recording and/or analyzing ECG for the purpose of providing diagnostic information to the physician. These devices have no telemetry link and have the disadvantage that the patient must wear surface electrodes and be connected to the recorder. This can particularly be a problem while the patient is sleeping. Also, since surface electrodes must be worn, patient compliance is an issue. Most patients are unwilling to wear surface electrodes for more than about three to four weeks. Devices are often worn for two to four weeks. If problems have occurred in the recording, it will not be noticed for quite some time.

### BRIEF SUMMARY OF THE INVENTION

Implantable medical devices and associated methods are disclosed. In one implementation, the implantable medical device comprises a conductive housing and a remote electrode that is mechanically coupled to the conductive housing by a lead body. An amplifier is electrically connected to the remote electrode and the conductive housing for providing a signal representative of a voltage difference between the remote electrode and the conductive housing. In some methods in accordance with the present invention, the implantable medical device is implanted in an implant site overlaying one half of a rib cage of a human body. The implantable medical device produces a signal representative of the voltage difference between the remote electrode and the conductive housing and the signal is transmitted to a receiver located outside the human body.

### BRIEF DESCRIPTION OF THE DRAWING

FIG. 1 is a schematic illustration showing a system for monitoring one or more physiological signals telemetered from an implantable medical device implanted in a human patient.

FIG. 2 is a plan view showing an implantable medical device that is implanted in a human body.

FIG. 3 is an isometric view showing a portion of a human body with an implantable medical device implanted therein.

FIG. 4 is an isometric view showing a left implant site disposed in the left half of the human body shown in the previous figure.

FIG. 5 is an isometric view showing a right implant site disposed in the right half of the human body shown in the previous figure.

FIG. 6 is a transverse cross-sectional view of a human body with an implantable medical device implanted therein.

FIG. 7 is a cross-sectional view showing an implantable medical device in accordance with an exemplary embodiment of the present invention.

FIG. 8 is an additional cross sectional view of the implantable medical device shown in the previous figure.

FIG. 9 is an axial view of a lead assembly in accordance with an exemplary embodiment of the present invention.

FIG. 10 is a block diagram of an implantable medical device in accordance with an exemplary embodiment of the present invention.

FIG. 11 is a block diagram of an implantable medical device in accordance with an additional exemplary embodiment of the present invention.

FIG. 12 is a block diagram of an implantable medical device that is capable of producing a first signal that is representative of respiration and a second signal that is representative of ECG.

FIG. 13 is a flowchart illustrating an exemplary method in accordance with the present invention.

### DETAILED DESCRIPTION

The following detailed description should be read with reference to the drawings in which similar elements in different drawings are numbered the same. The drawings, which are not necessarily to scale, depict illustrative embodiments and are not intended to limit the scope of the invention.

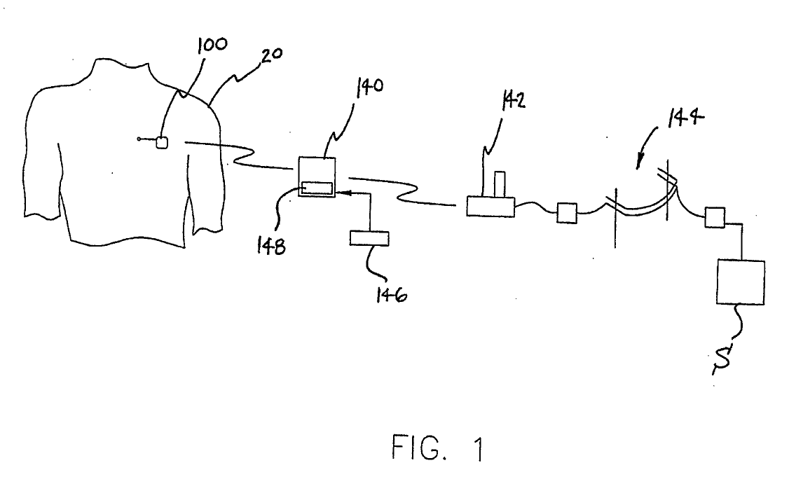

FIG. 1 is a schematic illustration showing a system for monitoring one or more physiological signals telemetered from implantable medical device 100 implanted in a human patient 20. In this illustrative embodiment, the system measures physiological signals such as ECG, pressure and/or temperature, and transmits (e.g., wirelessly) the waveforms of these signals to repeater 140 worn by or kept near patient 20. Repeater 140 receives the transmitted signals from implantable medical device 100 and retransmits (e.g., wirelessly) the signals to receiver/analyzer/storage buffer, RASB 142. Implantable medical device 100, repeater 140 and RASB 142 allow patient 20 to be monitored when lying in bed sleeping or going about normal daily activities. The RASB 142 may transmit the physiological data to a physician monitoring station S via a network 144. Network 144 may comprise various networks without deviating from the spirit and scope of the present invention. Examples of networks that may be suitable in some applications include the Internet and modem communication via telephone lines. Various communication techniques are described in the following U.S. Pat. Nos. 5,113,869; 5,336,245; 6,409,674; 6,347,245; 6,577,901; 6,804,559; 6,820,057. The entire disclosures of the above-mentioned U.S. Patents are hereby incorporated herein by reference. Various communication techniques are described in the following U.S. Patent Applications: US2002/0120200 and US2003/0074035. The entire disclosures of the above-mentioned U.S. Patent Applications are also hereby incorporated herein by reference.

Implantable medical device 100 may be dedicated to patient monitoring, or it may alternatively include a therapeutic function (e.g., pacing, defibrillation, etc.) as well. Repeater 140 may comprise a barometric pressure sensor 146 that measures barometric pressure and communicates the measurement to computing device 148. Computing device 148 subtracts barometric pressure from pressure measured by implantable medical device 100 to provide a gauge pressure measurement of internal body pressure. This gauge pressure signal is then retransmitted by repeater 140 to RASB 142, or it may be communicated back to a medical device implanted in patient 20 to aid in controlling delivery of a therapy. The therapeutic function may be contained within a separate implantable device that is in communication with repeater 140 or/and implantable medical device 100. This therapeutic function may be controlled in part by information derived separately or in combination from repeater 140 or/and medical device.

Implantable medical device 100 may transmit signals in real time or pseudo real time (slightly delayed from real time). If the transmissions occur in true real time, and if the waveforms were to be transmitted either continuously or frequently, in order to achieve satisfactory battery life, the transmitter may employ a modulation scheme such as Pulse Interval Modulation (PIM) and use a relatively low transmit carrier frequency (for example, tens or hundreds of kHz). Another approach to conserving power might be to process the signals within the medical device to extract the useful information. If the volume of data comprising the useful information is much less than the signals from which it was derived, the useful information may then be stored for later transmission, or it may then be transmitted in real time or pseudo real time to a receiver located outside the body. One limitation that is apparent in the Medtronic REVEAL device (Minneapolis, Minn.) is that the device often fills memory with false positive strips of what it perceives to be aberrant rhythms. By transmitting the raw data to a processor located outside the body, the useful information contained in the signals can be more precisely extracted

A limitation of using PIM and a low carrier frequency is that the transmit range is relatively short and the signal transmission is subject to interference. This limitation can be overcome by locating repeater 140 in close proximity to implantable medical device 100. This can be accomplished by wearing repeater 140 in close proximity to implantable medical device 100 by attaching it to lanyard or clip, or by securing it to a strap or elastic garment worn on patient 20.

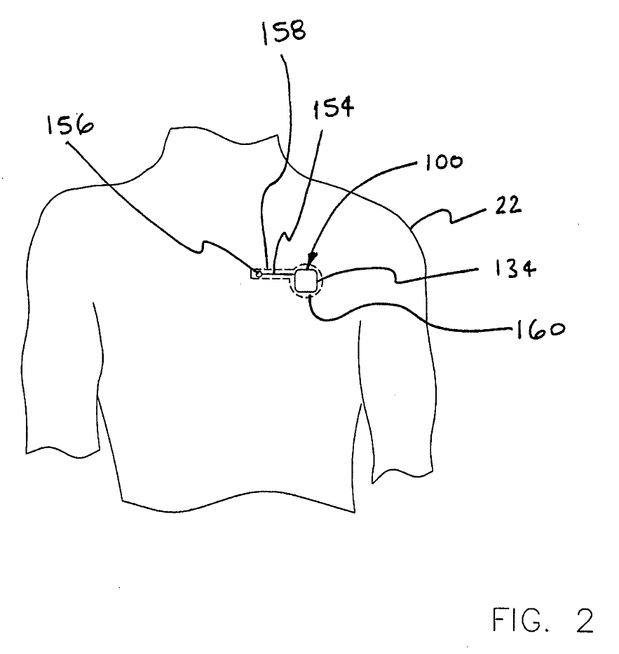

FIG. 2 is a plan view showing an implantable medical device 100 that is implanted in a human body 22. In the embodiment of FIG. 2, implantable medical device 100 comprises a housing 134, a lead body 154, and a remote electrode 156. With reference to FIG. 2, it will be appreciated that housing 134 is disposed in a pocket 160 that has been formed in the tissues of human body 22. With continuing reference to FIG. 2, it will be appreciated that remote electrode 156 is disposed in a channel 158 that has been formed in the tissue of human body 22. In some methods in accordance with the present invention, pocket 160 and channel 158 are formed within a pre-selected implant site inside human body 22.

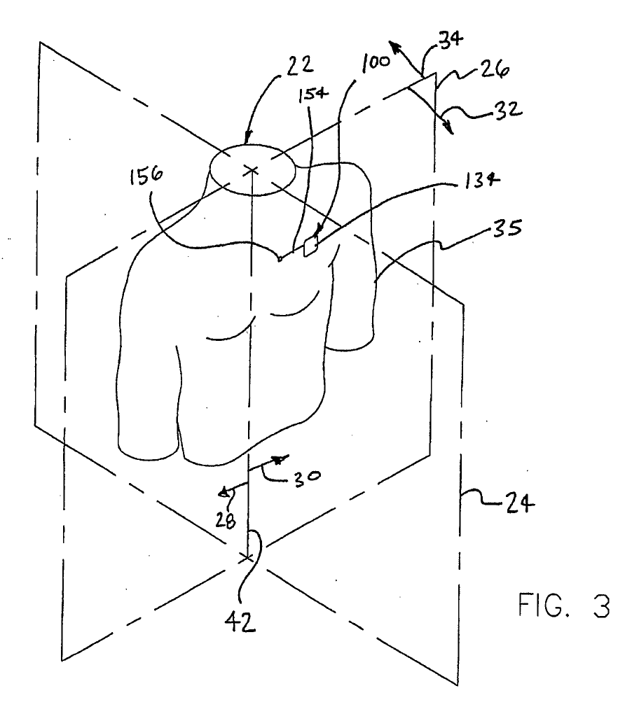

FIG. 3 is an isometric view showing a portion of a human body 22 with an implantable medical device 100 implanted therein. In FIG. 3, a central sagital plane 24 and a frontal plane 26 are shown intersecting human body 22. In the embodiment of FIG. 3, central sagital plane 24 and frontal plane 26 intersect one another at a median axis 42 of human body 22. With reference to FIG. 3, it will be appreciated that central sagital plane 24 bisects human body 22 into a right half 28 and a left half 30. Also with reference to FIG. 3, it will be appreciated that frontal plane 26 divides human body 22 into an anterior portion 32 and a posterior portion 34. In the embodiment of FIG. 3, central sagital plane 24 and a frontal plane 26 are generally perpendicular to one another.

With reference to FIG. 3, it will be appreciated that implantable medical device 100 is implanted in tissue proximate a left arm 35 of human body 22. In the embodiment of FIG. 3, implantable medical device 100 comprises a housing 134, a remote electrode 156 and a lead body 154 that mechanically couples remote electrode 156 to housing 134.

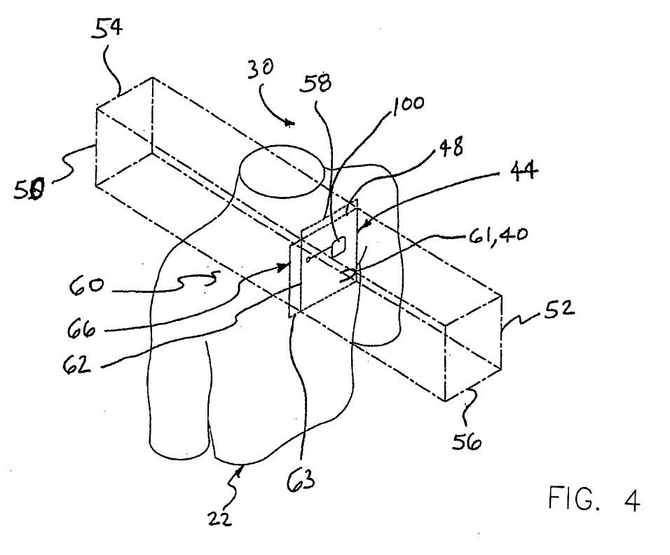

FIG. 4 is an isometric view showing a left implant site 44 disposed in the left half 30 of the human body 22 shown in the previous figure. With reference to FIG. 4, it will be appreciated that an implantable medical device 100 is disposed in the left implant site 44. As shown in FIG. 4, left implant site 44 may be defined by reference to a plurality of planes. A first sagittal plane 50 is shown contacting a left-most extent 62 of a sternum 66 of human body 22. A second sagittal plane 52 is shown contacting a left-most extent 61 of a rib cage 40. In the embodiment of FIG. 4, left implant site 44 extends laterally between first sagittal plane 50 and second sagittal plane 52. A superior transverse plane 54 is shown contacting a lower surface 48 of a left clavicle 58 of human body 22. An inferior transverse plane 56 is shown contacting a lower extent 63 of sternum 66. In the embodiment of FIG. 4, left implant site 44 extends between superior transverse plane 54 and inferior transverse plane 56. Some methods in accordance with the present invention, include the step of implanting implantable medical device 100 within left implant site 44. In some methods in accordance with the present invention, implantable medical device 100 is implanted between the skin 60 of the human body 22 and a front extent of rib cage 40.

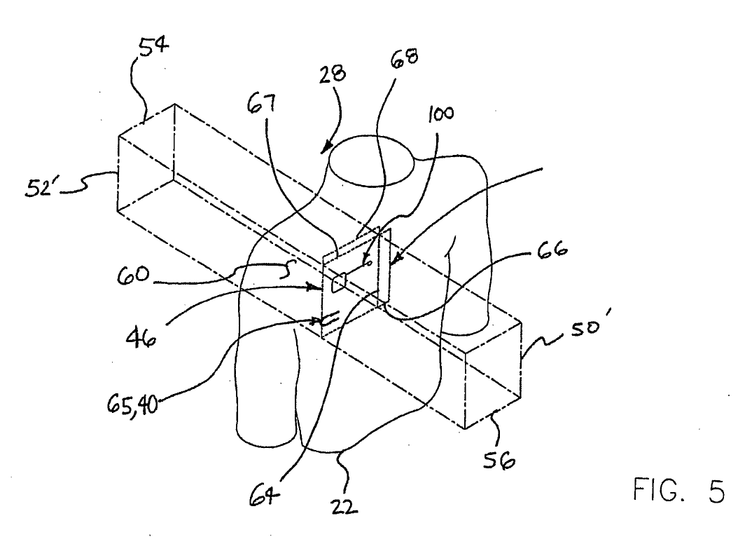

FIG. 5 is an isometric view showing a right implant site 46 disposed in the right half 28 of the human body 22 shown in the previous figure. With reference to FIG. 5, it will be appreciated that an implantable medical device 100 is disposed in the right implant site 46. As shown in FIG. 5, right implant site 46 may be defined by reference to a plurality of planes. A first sagittal plane 50′ is shown contacting a right-most extent 64 of a sternum 66 of human body 22. A second sagittal plane 52′ is shown contacting a right-most extent 65 of a rib cage 40. In the embodiment of FIG. 5, right implant site 46 extends laterally between first sagittal plane 50′ and second sagittal plane 52′. A superior transverse plane 54 is shown contacting a lower surface 67 of a right clavicle 68 of human body 22. An inferior transverse plane 56 is shown contacting a lower extent sternum 66. In the embodiment of FIG. 5, right implant site 46 extends between superior transverse plane 54 and inferior transverse plane 56. Some methods in accordance with the present invention, include the step of implanting implantable medical device 100 within right implant site 46. In some methods in accordance with the present invention, implantable medical device 100 is implanted between the skin 60 of the human body 22 and a front extent of rib cage 40.

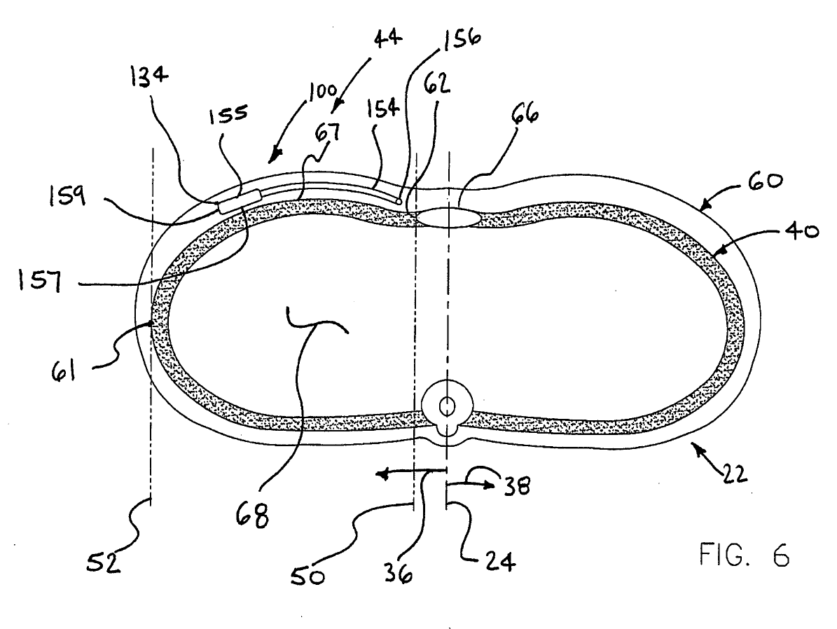

FIG. 6 is a transverse cross-sectional view of a human body 22 with an implantable medical device 100 implanted therein. The skin 60 and rib cage 40 of human body 22 are visible in this cross-sectional view. With reference to FIG. 6, it will be appreciated that implantable medical device 100 is disposed in a left implant site 44 of human body 22. Central sagital plane 24 is also shown in FIG. 6. With reference to FIG. 6, it will be appreciated that central sagital plane 24 bisects rib cage 40 into a right half 38 and a left half 36. With reference to FIG. 6, it will be appreciated that left implant site 44 generally overlays left half 36 of rib cage 40. In FIG. 6, implantable medical device 100 is show positioned outside of rib cage 40.

With reference to FIG. 6, it will be appreciated that implantable medical device 100 is disposed between skin 60 of human body 22 and a frontal extent 67 of the rib cage 40 of human body 22. In the embodiment of FIG. 6, left implant site 44 extends between a first sagittal plane 50 and a second sagittal plane 52. In FIG. 6, first sagittal plane 50 is shown contacting a left-most extent 62 of a sternum 66 of human body 22. Also in FIG. 6, second sagittal plane 52 is shown contacting a left-most extent 61 of rib cage 40.

In the embodiment of FIG. 6, implantable medical device 100 comprises a housing 134, a lead body 154, and a remote electrode 156. Housing 134 comprises a first major side 155 and a second major side 157. In some useful embodiments of the present invention, first major side 155 and a second major side 157 each comprise a conductive outer surface. When the is the case, first major side 155 and a second major side 157 may both make electrical contact with body tissues. In the embodiment of FIG. 6, first major side 155 has a greater surface area than a minor side 159 of housing 134.

In FIG. 6, lead body 154 is shown assuming a generally curved shape. In some useful embodiments of the present invention, lead body 154 has sufficient lateral flexibility to allow lead body 154 to conform to the contour of left implant site 44. Also in some useful embodiments of the present invention, lead body 154 has sufficient lateral flexibility to allow lead body 154 to flex in compliance with muscle movements of human body 22. With reference to FIG. 6, it will be appreciated that lead body 154 does not extend into a chest cavity 68 of human body 20. Accordingly, it will be appreciated that lead 154 does not extend into a cavity of the heart of human body 20.

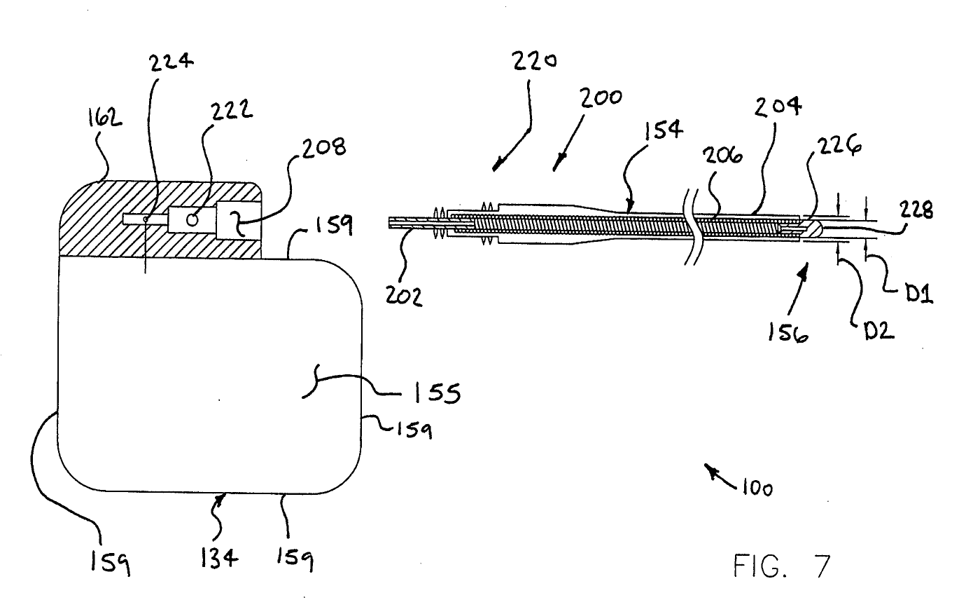

FIG. 7 is a cross-sectional view showing an implantable medical device 100 in accordance with an exemplary embodiment of the present invention. Implantable medical device 100 comprises a conductive housing 134, a header 162, and a lead assembly 200. Lead assembly 200 comprises a remote electrode 156 and a connector pin 202. Remote electrode 156 and connector pin 202 are mechanically coupled to one another by a lead body 154 of lead assembly 200. Lead body 154 comprises a coiled conductor 206 and an outer sheath 204. In some useful embodiments, outer sheath comprises a flexible material. Examples of flexible materials that may be suitable in some applications include silicone rubber and polyurethane.

Remote electrode 156 and connector pin 202 are also electrically connected to one another by coiled conductor 206. Coiled conductor 206 may comprise one or more filars wound in a generally helical shape. For example, coiled conductor 206 may comprise four helically wound filars. Remote electrode 156 may comprise various materials without deviating from the spirit and scope of the present invention. Examples of materials that may be suitable in some applications include stainless steel, Elgiloy, MP-35N, titanium, gold and platinum. Remote electrode 156 may also comprise a coating. Examples of coatings that may be suitable in some applications include carbon black, platinum black, and iridium oxide.

Header 162 defines a socket 208 that is dimensioned to receive a connecting portion 220 of lead assembly 200. Remote electrode 156 may be detachably attached to conductive housing 134 by inserting connecting portion 220 of lead assembly 200 into socket 208. In the embodiment of FIG. 7, a set screw 222 is disposed in a threaded hole defined by header 162. Set screw may be used to selectively lock connecting portion 220 of lead assembly 200 in socket 208. An electrical contact 224 is also shown in FIG. 7. Electrical contact 224 may make contact with connector pin 202 when connecting portion 220 of lead assembly 200 is disposed in socket 208.

Housing 134 comprises a first major side 155 and an opposing second major side. In some useful embodiments of the present invention, the first major side and the second major side of housing 134 each comprise a conductive outer surface. In the embodiment of FIG. 7, first major side 155 has a greater surface area than minor sides 159 of housing 134.

In the embodiment of FIG. 7, remote electrode 156 comprises a generally cylindrical body portion 226 having a generally circular lateral cross section. With reference to FIG. 7 it will be appreciated that remote electrode 156 also comprises a general rounded tip portion 228. In the embodiment of FIG. 7, tip portion 228 has a generally hemispherical shape. As illustrated in FIG. 7, remote electrode 156 has a first diameter D1 and lead body 154 has a second diameter D2. With reference to FIG. 7, it will be appreciated that first diameter D1 is generally smaller than second diameter D2. In some applications, providing a remote electrode having a diameter smaller than that of an attached lead body may facilitate removal of the remote electrode from the human body.

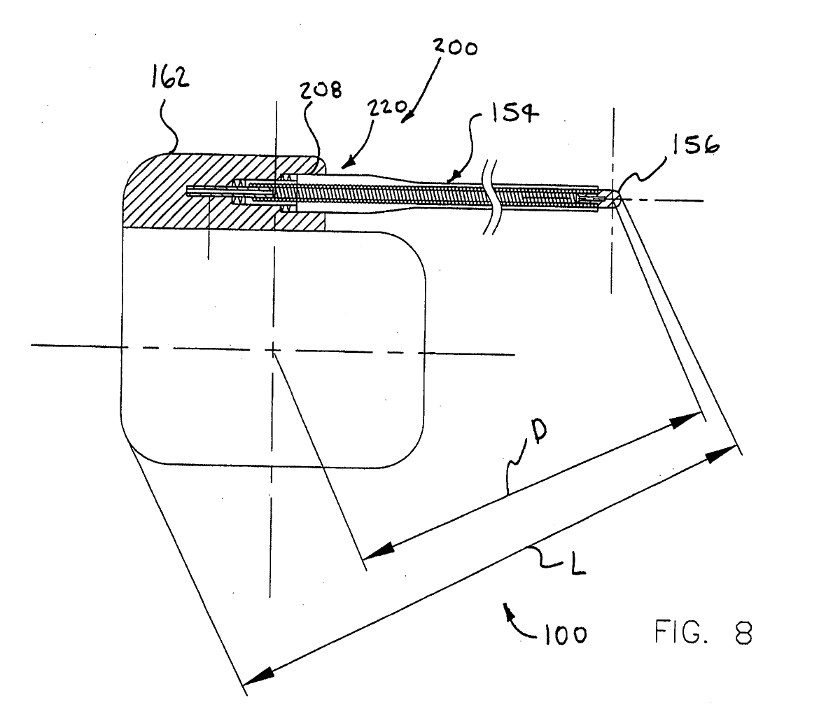

FIG. 8 is an additional cross sectional view of implantable medical device 100 shown in the previous figure. In the embodiment of FIG. 8, connecting portion 220 of lead assembly 200 is disposed in socket 208 defined by header 162. With reference to FIG. 8, it will be appreciated that remote electrode 156 and lead body 154 are both free of anchors. In some applications, providing a remote electrode that is free of anchors may facilitate removal of the remote electrode from the human body. Additionally, providing a lead body that is free of anchors may facilitate removal of the lead from the human body.

With reference to FIG. 8, it will be appreciated that lead body 154 separates remote electrode 156 and conductive housing 134 by a center-to-center distance D. In some useful embodiments, distance D is selected to be relatively large so that a voltage differential between conductive housing 134 and remote electrode 156 is relatively large. In some useful embodiments of the present invention, distance D is greater than about 4.0 centimeters and less than about 10.0 centimeters. In some particularly useful embodiments, distance D is greater than about 5.0 centimeters and less than about 7.0 centimeters.

With continuing reference to FIG. 8, it will be appreciated that implantable medical device 100 has an overall length L. In some useful embodiments of the present invention, overall length L is selected so that conductive housing 134, remote electrode 156, and lead body 154 will all be received in an implant site overlaying one half of a rib cage of a human body. In some useful embodiments of the present invention, overall length L is greater than about 4.0 centimeters and less than about 13.0 centimeters. In some particularly useful embodiments, overall length L is greater than about 5.0 centimeters and less than about 10.0 centimeters.

Conductive housing 134 may comprise various materials without deviating from the spirit and scope of the present invention. Examples of materials that may be suitable in some applications include stainless steel, Elgiloy, MP-35N, titanium, gold and platinum. Conductive housing 134 may also comprise a conductive coating. Examples of conductive coatings that may be suitable in some applications include carbon black, platinum black, and iridium oxide. In the embodiment of FIG. 8, conductive housing 134 is free of insulating coatings so that the entire outer surface of conductive housing 134 is available to make electrical connection with body tissue. Embodiments of the present invention are possible in which a portion of conductive housing 134 is covered with an insulating coating, for example, PARYLENE.

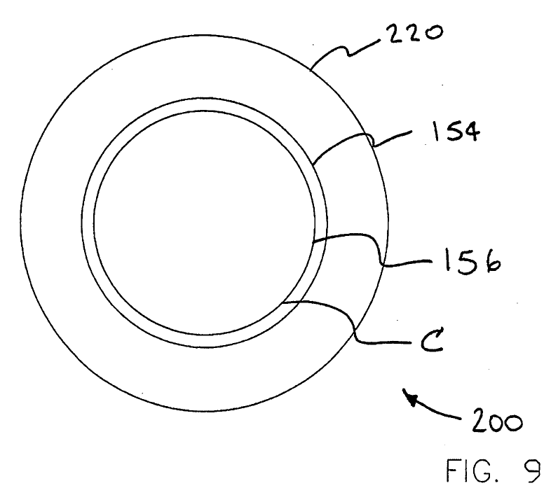

FIG. 9 is an axial view of lead assembly 200 shown in the previous figure. With reference to FIG. 9, it will be appreciated that remote electrode 156, lead body 154, and connecting portion 220 are all generally circular in cross section. In some applications, providing a remote electrode having a circular transverse cross-section may facilitate removal of the remote electrode from the human body. Additionally, providing a lead body having a circular transverse cross-section may facilitate removal of the lead from the human body. As illustrated in FIG. 9, remote electrode 156 has a circumference C. In some useful embodiments of the present invention, remote electrode 156 comprises a conductive surface along it's entire circumference C.

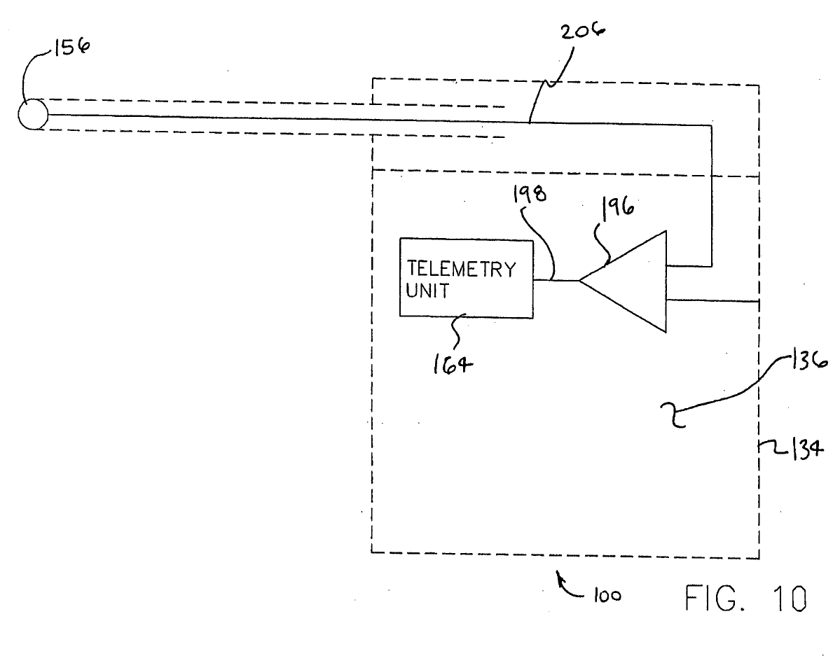

FIG. 10 is a block diagram of an implantable medical device 100 in accordance with an exemplary embodiment of the present invention. Implantable medical device 100 of FIG. 10 comprises a conductive housing 134 defining a cavity 136. In FIG. 10, an amplifier 196 is shown disposed in a cavity 136. A remote electrode 156 is electrically connected to amplifier 196 via a conductor 206. Amplifier 196 is also electrically connected to conductive housing 134. In the embodiment of FIG. 10, amplifier 196 is capable of detecting a voltage difference between conductive housing 134 and remote electrode 156. Amplifier 196 is also capable of producing a signal 198 that is representative of the voltage difference between conductive housing 134 and remote electrode 156. In FIG. 10, a telemetry unit 164 is shown connected to amplifier 196. In some useful embodiments of the present invention, implantable medical device 100 is disposed inside a human body and telemetry unit 164 is capable of transmitting signal 198 to a receiver located outside of the body.

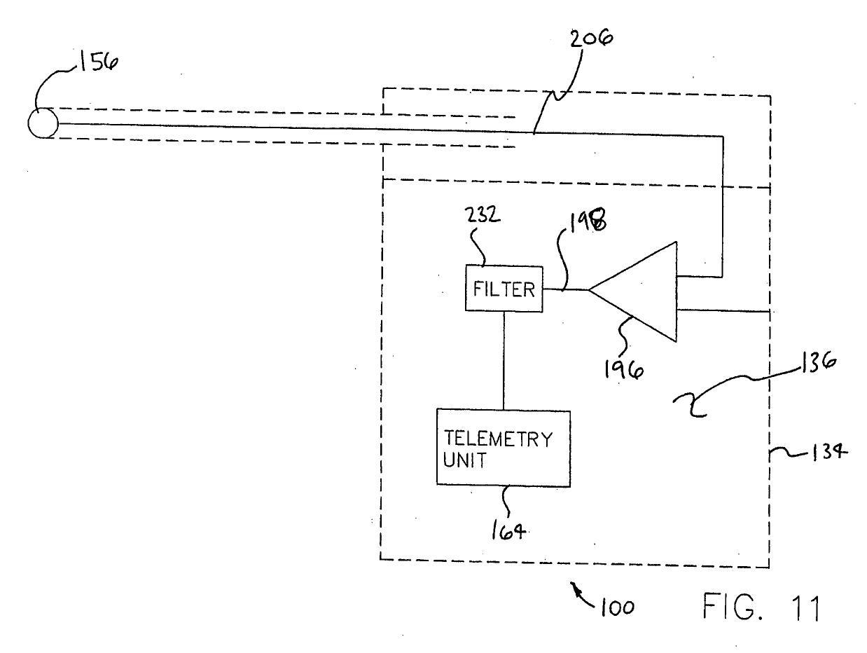

FIG. 11 is a block diagram of an implantable medical device 100 in accordance with an additional exemplary embodiment of the present invention. Implantable medical device 100 of FIG. 11 comprises a conductive housing 134 that is electrically connected to an amplifier 196. In the embodiment of FIG. 11, amplifier 196 is disposed within a cavity 136 defined by conductive housing 134. A remote electrode 156 is electrically connected to amplifier 196 via a conductor 206. In the embodiment of FIG. 11, amplifier 196 is capable of detecting a voltage difference between conductive housing 134 and remote electrode 156. Amplifier 196 is also capable of producing a signal 198 that is representative of the voltage difference between conductive housing 134 and remote electrode 156.

In the embodiment of FIG. 11, a filter 232 is electrically connected to amplifier 196. Filter 232 may be capable of filtering signal 198. Filter 232 may comprise, for example, a band-pass filter. When this is the case, filter 232 may pass a portion of signal 198 having frequency's between about 0.5 Hz and about 80.0 Hz. Filter 232 is electrically connected to a telemetry unit 164. In some useful embodiments of the present invention, implantable medical device 100 is disposed inside a human body and telemetry unit 164 is capable of transmitting at least a portion of signal 198 to a receiver located outside of the body.

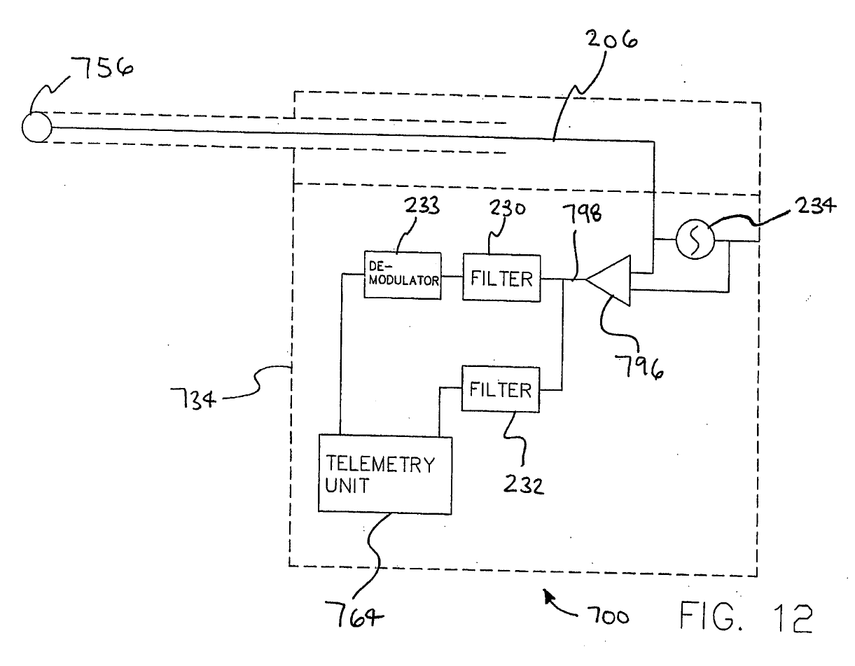

FIG. 12 is a block diagram of an implantable medical device 700 that is capable of producing a first signal that is representative of respiration and a second signal that is representative of ECG. Implantable medical device 700 of FIG. 12 comprises a conductive housing 734 that is electrically connected to a current source 234. A remote electrode 756 is also electrically connected to current source 234 via a conductor 206. In the embodiment of FIG. 12, current source 234 provides a substantially constant current traveling between conductive housing 734 and remote electrode 756.

In the embodiment of FIG. 12, an amplifier 796 is arranged to detect a voltage difference between conductive housing 734 and remote electrode 756. Amplifier 796 is also capable of producing a signal 798 that is representative of the voltage difference between conductive housing 734 and remote electrode 756. In the embodiment of FIG. 12, a first filter 230 and a second filter 232 are both connected to amplifier 796.

First filter 230 may comprise, for example, a band-pass filter that passes a portion of signal 798 that is related to the respiration of a human patient. For example, first filter 230 may pass a portion of signal 798 having frequency's between about 0.2 Hz and about 2.0 Hz. A de-modulator 233 is provided for demodulating the respiration related portion of signal 798.

Second filter 232 may comprise, for example, a band-pass filter that passes a portion of signal 798 that is related to ECG. For example, second filter 232 may pass a portion of signal 798 having frequency's between about 0.2 Hz and about 80.0 Hz. First filter 230 and second filter 232 are both electrically connected to a telemetry unit 764. In some useful embodiments of the present invention, implantable medical device 700 is disposed inside a human body and telemetry unit 764 is capable of transmitting at least a portion of signal 798 to a receiver located outside of the body.

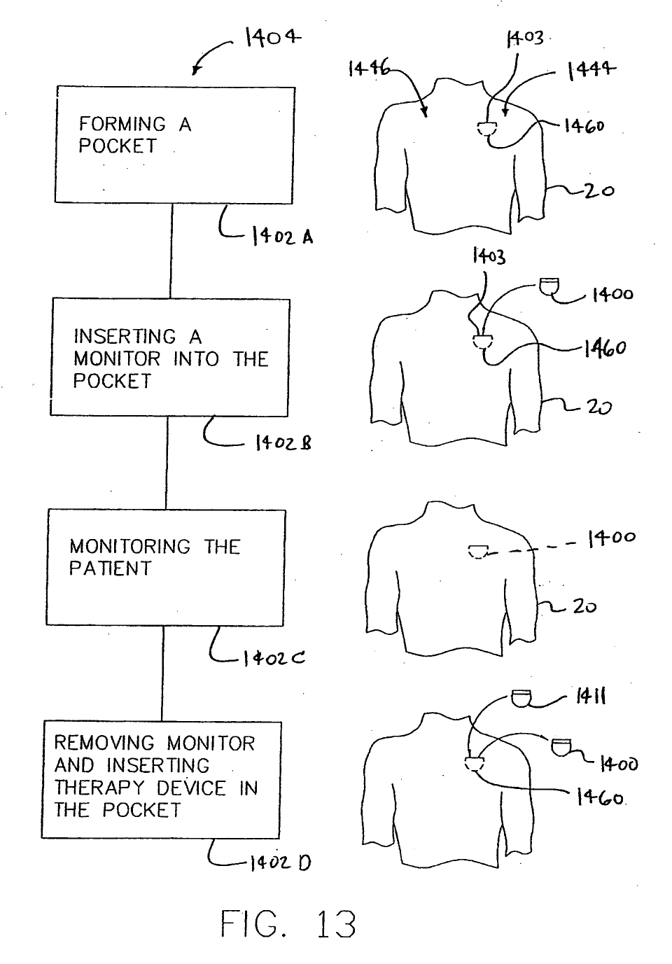

FIG. 13 shows a flowchart 1404 illustrating an exemplary method in accordance with the present invention. Block 1402A of flowchart 1404 illustrates the step of forming a pocket 1460 in a left implant site 1444 in the body of a patient 20. In should be noted that pocket 1460 may be formed in a right implant site 1446 of the body of patient 20 without deviating from the spirit and scope of the present invention. Pocket 1460 may be formed, for example, by making an incision 1403 with a cutting tool and pushing a blunt object through the incision 1403 to displace tissue and form pocket 1460. Pocket 1460 may also be formed by pushing gloved fingers through incision 1403.

Block 1402B of flowchart 1404 illustrates the step of inserting an implantable monitoring device 1400 in pocket 1460. Implantable monitoring device may comprise, for example, the implantable medical devices described herein. Implantable monitoring device 1400 may be inserted through incision 1403 so that the housing of implantable monitoring device 1400 is positioned within pocket 1460 adjacent to incision 1403. Incision 1403 may then be closed and the patient may be allowed to go about a normal daily routine.

Block 1402C of flowchart 1404 illustrates the step of monitoring the patient. Implantable monitoring device 1400 may detect various physiological parameters such as, for example, ECG, pressure and temperature. Implantable monitoring device 1400 may transmit (e.g., wirelessly) signals related to these parameters to a repeater worn by or kept near patient 20. Patient 20 may be monitored during normal daily activity for a period of weeks, months and/or years.

A method in accordance with the present invention may include, for example, the steps of placing an implantable monitoring device comprising a conductive housing and a remote electrode in a left implant site 1444 and detecting a voltage difference between the remote electrode and the conductive housing. This method may further include the step of producing a signal representative of the voltage difference between the remote electrode and the conductive housing. The signal may be transmitted to a receiver located outside the human body. Information obtained during the monitoring step may be analyzed to determine what type of implantable therapy device may be appropriate for patient 20.

Block 1402D of flowchart 1404 illustrates the steps of removing implantable monitoring device 1400 from pocket 1460 and inserting an implantable therapy device 1411 in pocket 1460. In some useful methods in accordance with the present invention, implantable monitoring device 1400 is removed from pocket 1460 and implantable therapy device 1411 is inserted in pocket 1460 during a single surgical procedure. In the embodiment of FIG. 13, implantable monitoring device 1400 and implantable therapy device 1411 have similar shapes and a similar in size.

Implantable therapy device 1411 may comprise various elements without deviating from the spirit and scope of the present invention. Examples of implantable therapy devices that may be suitable in some applications include pacemakers, defibrillators, and/or cardioverters. In some useful methods in accordance with the present invention, pocket 1460 is disposed in a location which will allow leads connected to implantable therapy device 1411 to travel through the vasculature of patient 20 to the heart of patient 20.

Those skilled in the art will recognize that the present invention may be manifested in a variety of forms other than the specific embodiments described herein. Accordingly, departures in form and detail may be made without departing from the spirit and scope of the present invention as described in the appended claims.

What is claimed is:

1. An implantable medical device, comprising:
a conductive housing having an interior;
electronic circuitry disposed in the interior of the housing;
the electronic circuitry being electronically connected to the conductive housing and to a remote electrode that is offset from the conductive housing; and
a lead body extending between the conductive housing and the remote electrode.

2. The implantable medical device of claim 1, wherein the lead body has sufficient lateral flexibility to allow relative motion between the remote electrode and the conductive housing when the implantable medical device is implanted in a human body.

3. The implantable medical device of claim 1, wherein the lead body has sufficient longitudinal stiffness to substantially maintain spacing between the remote electrode and the conductive housing.

4. The implantable medical device of claim 1, wherein the remote electrode comprises a generally cylindrical body portion.

5. The implantable medical device of claim 1, wherein the remote electrode comprises a rounded tip portion.

6. The implantable medical device of claim 5, wherein the remote electrode comprises a generally hemispherical tip portion.

7. The implantable medical device of claim 1, wherein the remote electrode is free of anchors for facilitating removal of the remote electrode from a body.

8. The implantable medical device of claim 1, wherein the lead body is free of anchors for facilitating removal of the lead body electrode from a body.

9. The implantable medical device of claim 1, wherein the remote electrode is conductive along an entire circumference thereof.

10. The implantable medical device of claim 1, wherein:
the remote electrode has a first maximum dimension;
the lead body has a second maximum dimension; and
the first maximum dimension is smaller than the second maximum dimension.

11. The implantable medical device of claim 1, wherein the electronic circuitry comprises a constant current source electrically connected to the conductive housing and the remote electrode.

12. The implantable medical device of claim 1, wherein the conductive housing comprises a first major side facing a first direction and a second major side facing a second direction.

13. The implantable medical device of claim 11, wherein the second direction is generally opposite the first direction.

14. The implantable medical device of claim 11, wherein the first major side and the second major side each comprise a conductive outer surface.

15. The implantable medical device of claim 1, wherein the lead body separates the remote electrode and the conductive housing by a center to center distance that is selected so that the conductive housing, the remote electrode, and the lead body will all be received in an implant site overlaying one half of a rib cage of a human body.

16. The implantable medical device of claim 14, wherein the implant site extends between a skin and a rib cage of the human body.

17. The implantable medical device of claim 14, wherein the implant site extends between a left-most extent of a sternum of the human body and a left-most extent of a rib cage of the human body.

18. The implantable medical device of claim 14, wherein the implant site extends between a lower-most surface of a clavicle of the human body and a lower-most extent of a sternum of the human body.

19. The implantable medical device of claim 14, wherein the pre-selected distance is greater than about 4.0 centimeters and less than about 1 0.0 centimeters.

20. The implantable medical device of claim 19, wherein the pre-selected distance is greater than about 5.0 centimeters and less than about 7.0 centimeters.
